(19) United States
(12) Patent Application Publication
(10) Pub. No.: US 2003/0105183 A1
(43) Pub. Date: Jun. 5, 2003

(54) Scented hot melt adhesives

(76) Inventor: Matthew L. Sharak, Franklin Park, NJ (US)

Correspondence Address: Cynthia L. Foulke, NATIONAL STARCH AND CHEMICAL COMPANY, 10 Finderne Avenue, Bridgewater, NJ, 08807-0500, US

(21) Appl. No.: 10/006,592

(22) Filed: Dec. 5, 2001

(51) Int. Cl. A61L 2/00

(52) U.S. Cl. 523/102

(57) ABSTRACT

Hot melt adhesives comprising an effective amount of a scented material having a closed cup flash point of greater than 100° F. The scented adhesive may preferably be an essential oil, most preferably an essential lavender oil.

### FIELD OF THE INVENTION

The present invention relates to scented adhesives. In particular, the invention relates to scented hot melt adhesives, to the use of a scented hot melt adhesive to prepare a disposable absorbent product and to disposable absorbent articles comprising scented hot melt adhesives.

### BACKGROUND OF THE INVENTION

Disposable absorbent products currently find widespread use in many applications. For example, diapers, training pants, incontinent garments, feminine hygiene pads, i.e., catamenial pads, sanitary napkins, panty liners, panty shields and the like, underarm shields and health care products such as surgical drapes or wound dressings. Such articles are designed to absorb body fluids, such as urine, menses, blood, perspiration and other excrements discharged by a body.

A typical disposable absorbent product generally comprises a composite structure including a fluid-permeable topsheet, a fluid absorbent core and a fluid-impermeable backsheet. These disposable absorbent products, when appropriate, usually include some type of fastening system for fitting the product onto the wearer.

Adhesives, often referred to as construction adhesives, are generally used to join the different parts of the disposable absorbent product together. Hot melt adhesives are preferably used for this purpose since such adhesives allow for cost and time efficient manufacturing since there is no evaporation step necessary as is the case for water-based or solvent-based adhesive systems.

While use of fragrances for odor control in sanitary disposable products, such as diapers and feminine hygiene products, has been addressed in the art, the incorporation of fragrances within the hot melt adhesive component used to construct such products has proved difficult. Effective perfuming of hot melt adhesives, i.e., wherein the fragrance remains stable during production, during storage prior to use, and is still available for delivery after the adhesive has been used, has been difficult to achieve and a need remains in the art for a fragrant hot melt adhesive that can be used to prepare disposable absorbent articles, in particular disposable absorbent garments such as diapers.

### SUMMARY OF THE INVENTION

It has now been discovered that certain scented compositions may be added to hot melt adhesives to provide a scent or fragrance to nonwoven fabrics.

This invention is directed to hot melt adhesives having contained therein molecules of at least one fragrance composition capable of evolving from said adhesive into the environment proximate said adhesive.

One aspect of the invention is directed to a hot melt adhesive comprising a scented material. The scented material preferably has a closed cup flash point of greater than 100° F., preferably greater than about 200° F. In a preferred embodiment, the scented material is an essential oil.

Another aspect of the invention is directed to a disposable absorbent article of manufacture comprising a liquid-permeable topsheet, a liquid-impermeable backsheet, a fluid-absorbent core material positioned between the topsheet and the backsheet and a hot melt adhesive containing a scented material. A particularly preferred aspect of the invention is directed to disposable absorbent garments such as, for example, diapers for use in infants and toddlers, adult incontinent pads and feminine hygiene products.

Still another aspect of the invention is directed to a process for bonding a tissue or a nonwoven substrate to similar or dissimilar substrates in the construction of a disposable absorbent product comprising applying to at least one substrate a molten hot melt adhesive composition and bonding the substrates together, wherein the hot melt adhesive comprises a scented material.

### DETAILED DESCRIPTION OF THE INVENTION

The invention provides fragranced compositions, fragranced adhesive compositions and a process for the effective perfuming of hot melt adhesives. The fragranced compositions may be added to hot melt adhesives without the a requirement for encapsulation or other protective means, and are capable of remaining stable in the adhesives prior to and after use without significant loss of fragrance.

The term effective perfuming means the fragrance remains stable during production of the adhesive, during storage of the adhesive prior to use and is still available for delivery after the adhesive has been used. By delivery means the scent evolves from the adhesive into the environment proximate to the adhesive. By stable is meant that the fragrance remains noticeable following manufacture of the adhesive, following storage of the adhesive, following manufacture of an article formed using the adhesive (e.g., a diaper), during storage of the manufactured article and during use of the manufactured article by the end user.

The terms perfume, fragrance, aroma, odor and scent are used interchangeably throughout this application and mean a composition comprising one or more fragrance materials, and optionally a solvent, formulated to have certain useful or perceived fragrance characteristics. These terms are used in their ordinary sense to refer to and include any fragrant substance or mixture of substances including natural (i.e., obtained by extraction of flower, herb, blossom or plant), artificial (i.e., mixture of natural oils or oil constituents) and synthetically produced substance and odoriferous compounds.

In most cases fragrance compositions are formulated to have a fragrance generally considered at least inoffensive and preferably pleasing to intended users of the composition or user of the article prepared with the adhesive. Fragrance compositions may also be used for imparting a desired odor to the skin and/or any product for which an agreeable odor is indispensable or desirable. Fragrance compositions are also used in products that would normally have an unattractive or offensive odor to mask the odor and produce an odor that is less unattractive or offensive. The (pleasing) fragrance characteristics may be the main function of the product in which the fragrance composition has been incorporated or may be ancillary to the main function of the product.

In one embodiment of the invention, a disposable absorbent product is provided, which disposable absorbent product comprises (1) a liquid-permeable topsheet, (2) a liquid-impermeable backsheet, which topsheet may be attached to the backsheet, (3) an absorbent structure positioned between the topsheet and the backsheet, and (4) an adhesive having desired fragrant properties.

The absorbent structure will typically comprise a nonwoven fabric. A nonwoven fabric is defined as an interlocking fiber network characterized by flexibility, porosity and integrity. The individual fibers used to compose the nonwoven fabric may be synthetic, naturally occurring, or a combination of the two. The individual fibers may be mechanically, chemically, or thermally bonded to each other. Nonwovens are used commercially for a variety of applications including insulation, packaging, household wipes, surgical drapes, medical dressings, and in disposable articles such as diapers, adult incontinent products and sanitary napkins. Tissue is a closely related material in which the individual fibers may or may not be chemically bonded to one another.

The adhesives according to the invention may be formulated for use as, e.g., positioning adhesives, core adhesives or elastic adhesives, and are particularly suitable for use in the manufacture or articles, including but not limited to disposable absorbent products, such as diapers, adult incontinent products, bed pads; sanitary napkins, and in other absorbent products, such as, bibs, wound dressings, and surgical capes or drapes, which are used to absorb a liquid, such as water and saline, and body liquids, such as urine, menses, and blood. The fragranced adhesive of the invention may be used to adhere the nonwoven or tissue to another substrate or component. The second substrate may be another nonwoven, tissue, or an unrelated material.

The adhesive may be used to attach the topsheet to the backsheet. Alternatively, the adhesive may be used to adhere either the topsheet or the backsheet to other components of the disposable absorbent product, such as tissue layers, leg flaps, fastening ears, tapes, or tabs, or other components typically used to construct a disposable absorbent product that are well known to one skilled in the art.

Those skilled in the art will recognize materials suitable for use as the topsheet and backsheet.

Exemplary of materials suitable for use as the topsheet are liquid-permeable materials, such as spunbonded polypropylene or polyethylene having a basis weight of from about 15 to about 25 grams per square meter.

Backsheets often used in disposable absorbent products are generally prepared from liquid-impermeable materials which function to contain liquids, such as water, urine, menses, or blood, within the absorbent core of the disposable absorbent product and to protect bedding and/or a wears' outer garments from soiling. Materials useful as a backsheet in a disposable absorbent product are generally impermeable to liquid but are permeable to vapor. Examples are liquid-impervious materials such as polyolefin films, e.g., polypropylene and polyethylene, as well as vapor-pervious materials, such as microporous polyolefin films, sometimes referred to as breathable films.

A particularly desirable backsheet material is a film comprising a polyolefin polymer such as a linear low density polyethylene and a filler. As used herein a “filler” is meant to include particulates and other forms of materials which can be added to the film polymer extrusion blend and which will not chemically interfere with or adversely affect the extruded film but which are able to be uniformly dispersed throughout the film. When the film is stretched during processing, the filler generally causes a network of holes to be formed in the film. Such holes are generally small enough to prevent the passage of a liquid, but are generally large enough to allow vapor to pass through the holes. Generally the fillers will be in particulate form and usually will have somewhat of a spherical shape with average particle sizes in the range of about 0.1 to about 7 microns. Both organic and inorganic fillers may be used in the practice of the invention provided that they do not interfere with the film formation process. Examples of fillers include calcium carbonate (CaCO₃), various kinds of clay, silica (SiO₂), alumina, barium sulfate, sodium carbonate, talc, magnesium sulfate, titanium dioxide, zeolites, aluminum sulfate, cellulose-type powders, diatomaceous earth, magnesium sulfate, magnesium carbonate, barium carbonate, kaolin, mica, carbon, calcium oxide, magnesium oxide, aluminum hydroxide, pulp powder, wood powder, cellulose derivatives, chitin and chitin derivatives.

Fragrance compositions which may be incorporated into hot melt adhesives in accordance with the invention are fragrances that have a closed cup flash point greater than about 100° F., preferably greater than about 150° F., more preferably greater than about 200° F. as determined by ASTM method D93-00 (Flash Point by Pensky-Martens Closed Cup Tester). Such fragrances are commercially available. An example of a fragrance which has closed cup flash point of greater than 200° F. is “Spicy Oriental Amber Powder” available from Quest (Quest Fragrance Q-26257),

Any fragrance or fragrances having a flashpoint greater that 100° F. may be used in the practice of the invention. In one embodiment, the fragrance is an essential oil. Essential oils provide a pleasing relaxing smell or aroma and, in may instances, a skin health benefit. Essential oils are highly concentrated, volatile liquids originating from a botanical source. A typical essential oil is a complex mixture of alcohols, aldehydes, esters, ketones, oxides, phenols and terpenes.

Essential oils can be extracted by steam distillation, and other techniques known in the art, from a variety of plant components, including, but not limited to, roots, leaves, bark, flowers and pulp. The majority of the oil-producing plants are represented by 90 species, and these are derived from about 30 different plant families.

Essential oils which may be used in the practice of the invention include but are not limited to achillea oil, anise oil, balm mint oil, basil oil, bee balm oil, bergamot oil, birch oil, bitter almond oil, bitter orange oil, calendula oil, California nutmeg oil, caraway oil, cardamom oil, chamomile oil, cinnamon oil, clary oil, cloveleaf oil, clove oil, coriander oil, cypress oil, eucalyptus oil, fennel oil, gardenia oil, geranium oil, ginger oil, grapefruit oil, hops oil, hyptis oil, indigo bush oil, jasmine oil, juniper oil, kiwi oil, laurel oil, lavender oil, lemongrass oil, lemon oil, linden oil, lovage oil, mandarin orange oil, matricaria oil, musk rose oil, nutmeg oil, olibanum, orange flower oil, orange oil, patchouli oil, pennyroyal oil, peppermint oil, pine oil, pine tar oil, rose hips oil, rosemary oil, rose oil, rue oil, sage oil, sambucus oil, sandalwood oil, sassafras oil, silver fir oil, spearmint oil, sweet marjoram oil, sweet violet oil, tar oil, tea tree oil, thulasia oil, thyme oil, turmeric oil, wild mint oil, yarrow oil, ylang ylang oil, and the like, as well as mixtures thereof.

A particularly preferred essential oil for use in the practice of the invention is lavender oil. Lavender belongs to the Lavandula genus which is a member of the Labiatae family, along with thyme, mint, rosemary, sage, and other herbs. About 20 different species of lavender exists including L. officinalis or angustifolia (English Lavender), L. dentata (French or fringed Lavender), L. stoechas (Spanish Lavender), L. spica, L. viridis (green lavender), L. lanata (wooly lavender), L. pinnata, L. multifida, and L. x heterophylla “Goodwin Creek”. L. angustifolia is the lavender species most often used as the source of essential lavender oil within the fragrance industry, and is a preferred essential oil for use in the practice of the invention.

It will be recognized that while the aroma of, e.g., an essential lavender oil may correlate to the perceived aroma of the living plant, the oil reflects the composition of volatiles and semi-volatiles present in the plant, with molecular transformations/rearrangements and degration products resulting from the high temperatures and harsh conditions prevailing during the distillation process. In general, the scent associated with the lavender plant is dominated by terpenes and sesquiterpenes, while the scent associated with the lavender oil distilled therefrom contains mostly alcohols and esters, e.g., linalool and linalyl acetate.

For a desired fragrance to be useful in the practice of the invention, it may be necessary to modify the fragrance by, for example, modifying the amounts and type of raw materials or by adding other ingredients so as to increase or otherwise raise the flash point to the required threshold. This is particularly true when the adhesive is to comprise an essential oil, since many essential oils exhibit closed cup flash points well below 100° F. making them unsuitable for use in the practice of the invention.

Various methods of modifying the fragrances, including essential oils, are within the skill of the art. Such modifications include modifying the amount and/or type of the fragrant raw materials comprising the essential oil, such as by substitution, functionalization, derivatization, forming adducts thereof and the like. Modification of the amount of raw materials contributing to the fragrant composition including modifying the amounts and type of isomers, e.g., the linalool (3,7-dimethyl-1,6-octadien-3-ol) component of the fragrant composition may be use as a racemic mixture or as each optical isomer alone, or may be replaced in whole or in part by a subsituted linalool, e.g. isobutyl linalool (3,7,9-trimethyl-1,6-decadien-3-ol). It will be appreciated that the components or raw materials making up the fragrant composition may be naturally occurring or modified, and may be synthetic. Combinations of natural, modified and/or synthetic components may be used.

The term esssential oil is defined broadly herein to include essential oils as distilled from the plant source, as well as derivatives, variations and mixtures thereof including synthetic forms. Mixtures of essential oils and synthetic forms of the oil or synthetic components thereof are also included. For example, it is possible to add to the essential oil distilled from the native lavender plant, synthetic chemical components thereof such as, for example, linalool and linalyl acetate, or add other types of essential oils, other aromic chemicals, synthetic forms thereof and modifying oils, such as mineral oils. Essential oil compositions that may be incorporated into the adhesives of the invention are manufactured by Essential Ingredients, Buford, Ga.

Fragrant materials, including modifications and compositions thereof, e.g., a modified essential oil composition, may be tested for closed cup flash point determined in accordance with ASTM method D93-00 (Flash Point by Pensky-Martens Closed Cup Tester). Materials and compositions having a closed cut flash point greater than about 100° F. may be advantageously used in the practice of the invention.

Other agents that provide a fragrance and/or a skin wellness benefit may also be used in the practice of the invention. Examples include cocoa butter (Theobroma cacao seed butter), Lanolin, Vitamin E and witch hazel (Hamamelis virginiana). These components, like the essential oils described herein, may be incorporated into a hot melt adhesive for use, e.g., as a construction adhesive in the manufacture of disposable absorbent articles.

The fragranced or fragrant composition(s) are added to the hot melt adhesive composition without the requirement for encapsulation or other protective means. The term non-encapsulated is used as conventional in the art and means that the fragrant material or composition is not enclosed in a protective cover or shell. The fragranced composition is formulated into the hot melt adhesive without the prior encapsulation thereof.

The fragrance compositions may be added to virtually any hot melt type adhesive. Any base polymer suitable for use in formulating hot melt adhesives, as are well known to those skilled in the art may be used in the practice of the invention. Such polymers include amorphous polyolefins, ethylene-containing polymers and rubbery block copolymers, as well as blends thereof. Hot melt adhesive compositions based on ethylene/vinyl acetate copolymers, isotactic or atactic polypropylene, styrene-butadiene, styrene-isoprene, or styrene-ethylene-butylene A-B-A or A-B-A-B block copolymers or mixtures thereof may be used. In addition to the base polymer, the hot melt adhesive compositions of the invention may also contain tackifiers, oils and/or waxes as well as conventional additives including stabilizers, anti-oxidants, pigments and the like.

In more detail, the fragrance compositions may be added to adhesives based on rubbery block copolymers. These polymers include the block or multi-block copolymers having the general configuration: A-B-A or A-B-A-B-A-B- wherein the polymer blocks A are non-elastomeric polymer blocks which, as homopolymers have glass transition temperatures above 20° C., while the elastomeric polymer blocks B are butadiene or isoprene or butadiene isoprene.

The non-elastomeric blocks may comprise homopolymers or copolymers of vinyl monomers such as vinyl arenes, vinyl pyridines, vinyl halides and vinyl carboxylates, as well as acrylic monomers such as acrylonitrile, methacrylonitrile, esters of acrylic acids, etc. Monovinyl aromatic hydrocarbons include particularly those of the benzene series such as styrene, vinyl toluene, vinyl xylene, ethyl vinyl benzene as well as dicyclic monovinyl compounds such as vinyl naphthalene and the like. Other non-elastomeric polymer blocks may be derived from alpha olefins, alkylene oxides, acetals, urethanes, etc.

The elastomeric block component of the copolymer may be isoprene or butadiene which may or may not be hydrogenated. Further, they may be linear or branched. Typical branched structures contain an elastomeric portion with at least three branches which can radiate out from a central hub or can be otherwise coupled together.

Hydrogenation may be either partial or substantially complete. Selected conditions may be employed for example to hydrogenate the elastomeric block while not so modifying the non-elastomeric blocks. Other conditions may be chosen to hydrogenate substantially uniformly along the polymer chain, both the elastomeric and non-elastomeric blocks thereof being hydrogenated to practically the same extent, which may be either partial or substantially complete.

Typical of the rubbery block copolymers useful herein are the polystyrene-polybutadiene-polystyrene, polystyrene-polyisoprene-polystyrene and e.g., polystyrene-poly-(ethylenebutylene)-polystyrene and polystyrene-poly-(ethylenepropylene)-polystyrene. These copolymers may be prepared using methods taught, for example, in U.S. Pat. Nos. 3,239,478; 3,427,269; 3,700,633; 3,753,936; and 3,932,327. Alternatively, they may be obtained from Kraton Chemical Co. under the trademarks Kraton 1101, 1102, 1107, 1650, 1652 and 1657; from Enichem under the Europrene Sol-T tradenames; and from Firestone under the tradename Stereon 840A. Adhesives based on rubbery block copolymers are described in U.S. Pat. Nos. 4,526,577, 4,944,993, 5,603,948 and others.

Ethylene containing polymers are also commonly used for disposable applications and can be fragranced by the addition thereto of the fragrance compositions in accordance with the teachings of the invention. The adhesive of the invention may comprise at least one ethylene copolymer, and may comprise a blend of two or more polymers. The term ethylene copolymer, as used herein, refers to homopolymers, copolymers and terpolymers of ethylene. Examples of ethylene copolymers include copolymers with one or more polar monomers which can copolymerize with ethylene, such as vinyl acetate or other vinyl esters of monocarboxylic acids, or acrylic or methacrylic acid or their esters with methanol, ethanol or other alcohols. Included are ethylene vinyl acetate, ethylene methyl acrylate, ethylene n-butyl acrylate, ethylene acrylic acid, ethylene methacrylate and mixtures and blends thereof. Random and block copolymers, as well as blends thereof may be used in the practice of the invention.

Other adhesive compositions may be prepared according to the invention using, as a base polymer, amorphous polyolefins or blends thereof. Amorphous polyolefins are made by the stereospecific polymerization of polypropylene. Suitable commercial products include Eastman's P 1010. Copolymers of amorphous polypropylene and ethylene, amorphous polypropylene and butene and amorphous polypropylene and hexene are suitable as a base polymer, as are terpolymers of propylene, butene and ethylene. Commercial examples include Rextac 2315 (copolymer of amorphous polypropylene and ethylene) available from Rexene, Rextac 2730 (copolymer of amorphous polypropylene and butene) also available from Rexene and Vestoplast 750 and 708 (terpolymers of amorphous propylene, butene and ethylene) available from Huls.

Blends of any of the above base materials, such as blends of ethylene n-butyl acrylate and ethylene vinyl acetate and ethylene vinyl acetate and atactic polypropylene may also be used to prepare hot melt adhesive compositions. In all cases, the adhesives may be formulated with tackifying resins, plasticizers, waxes and/or other conventional additives in varying amounts as are known to those skilled in the art and as required for particular formulations, e.g., a pressure sensitive adhesive formulation.

Preferred are low application temperature hot melt adhesive formulations, i.e., formulations that can be applied at temperatures below about 300° F., more preferably at about 250° F. and down to about 200° F. Low application temperature hot melt adhesives are commercially available from National Starch and Chemical Company, Bridgewater, N.J.

Tackifying resins useful in the adhesive compositions of this invention include hydrocarbon resins, synthetic polyterpenes, rosin esters, natural terpenes, and the like. More particularly, and depending upon the particular base polymer, the useful tackifying resins may include any compatible resins or mixtures thereof such as natural and modified rosins including, for example, as gum rosin, wood rosin, tall oil rosin, distilled rosin, hydrogenated rosin, dimerized rosin, and polymerized rosin; glycerol and pentaerythritol esters of natural and modified rosins, including, for example as the glycerol ester of pale, wood rosin, the glycerol ester of hydrogenated rosin, the glycerol ester of polymerized rosin, the pentaerythritol ester of hydrogenated rosin, and the phenolic-modified pentaerythritol ester of rosin; copolymers and terpolymers of natured terpenes, including, for example, styrene/terpene and alpha methyl styrene/terpene; polyterpene resins having a softening point, as determined by ASTM method E28-58T, of from about 80° C. to 150° C.; phenolic modified terpene resins and hydrogenated derivatives thereof including, for example, the resin product resulting from the condensation, in an acidic medium, of a bicyclic terpene and a phenol; aliphatic petroleum hydrocarbon resins having a Ball and Ring softening point of from about 70° C. to 135° C.; aromatic petroleum hydrocarbon resins and the hydrogenated derivatives thereof; and alicyclic petroleum hydrocarbon resins and the hydrogenated derivatives thereof. Mixtures of two or more of the above described tackifying resins may be required for some formulations.

Various plasticizing or extending oils are also present in the composition in amounts of 5% to about 30%, preferably 5 to 25%, by weight in order to provide wetting action and/or viscosity control. Even higher levels may be used in cases where block copolymer containing hydrogenated mid-block are employed as the adhesive base polymer. The above broadly includes not only the usual plasticizing oils but also contemplates the use of olefin oligomers and low molecular weight polymers as well as vegetable and animal oil and their derivatives. The petroleum derived oils which may be employed are relatively high boiling materials containing only a minor proportion of aromatic hydrocarbons (preferably less than 30% and, more particularly, less than 15% by weight of the oil). Alternatively, the oil may be totally non-aromatic. The oligomers may be polypropylenes, polybutenes, hydrogenated polyisoprene, hydrogenated polybutadiene, or the like having average molecular weights between about 350 and about 10,000. Vegetable and animal oils include glyceryl esters of the usual fatty acids and polymerization products thereof.

Various petroleum derived waxes may also be used in amounts less than about 15% by weight of the composition in order to impart fluidity in the molten condition of the adhesive and flexibility to the set adhesive, and to serve as a wetting agent for bonding cellulosic fibers. The term “petroleum derived wax” includes both paraffin and microcrystalline waxes having melting points within the range of 130° F. to 225° F. as well as synthetic waxes such as low molecular weight polyethylene or Fisher-Tropsch waxes.

An antioxidant or stabilizer may also be included in the adhesive compositions described herein in amounts of up to about 3% by weight. Among the applicable antioxidants or stabilizers are high molecular weight hindered phenols and multifunctional phenols such as sulfur and phosphorous-containing phenols. Representative hindered phenols include: 1,3,5-trimethyl 2,4,6-tris (3,5-di-tert-butyl-4-hydroxy-benzyl)benzene; pentaerythritol tetrakis-3(3,5-di-tert-butyl-4-hydroxyphenyl)-propionate; n-octadecyl-3,5-di-tert-butyl-4-hydroxyphenol)-propionate; 4,4′-methylenebis (2,6-tert-butylphenol); 4,4′-thiobis (6-tert-butyl-o-cresol); 2,6-di-tertbutylphenol; 6-(4-hydroxyphenoxy)-2,4-bis(n-octyl-thio)-1,3, 5-triazine; di-n-octadecyl 3,5-di-tert-butyl-4-hydroxy-benzyl-phosphonate; 2n-octylthio)-ethyl 3,5-di-tert-butyl-4hydroxy-benzoate and sorbitol hexa[3-(3,5-ditert-butyl-4-hydroxyphenyl)-propionate].

Other additives conventionally used in hot melt adhesives to satisfy different properties and meet specific application requirements also may be added to the adhesive composition of this invention. Such additives include, for example, fillers, pigments, flow modifiers, dyestuffs, which may be incorporated in minor or larger amounts into the adhesive formulation, depending on the purpose.

Hot melt adhesives may be prepared using techniques known in the art. Typically, the adhesive compositions are prepared by blending the components in the melt at a temperature of about 100° to 200° C. until a homogeneous blend is obtained, approximately two hours. Various methods of blending are known and any method that produces a homogeneous blend is satisfactory. The fragrance composition may be added, with stirring, any time during the preparation of the hot melt adhesive. The fragrance is added in an amount effective to be noticeable, and remain noticeable, to the user of the adhesive following cure and to the user of any end product manufactured using the scented adhesive of the invention.

The effectiveness of an essential oil to provide a fragrance may be determined by gathering organoleptic data from a panel presented with a series of absorbent article samples containing increasing concentrations of essential oil. The samples are then evaluated in a variety of environments, including but not limited to prior to wearing and during wearing. The panelists are asked a series of questions relating to their perceptions of the effectiveness of essential oil, at various add-on amounts, to provide an aroma.

As will be appreciated by the skilled artisan, the amount of fragrance incorpoated into the adhesive will depend on the origin, composition, purity, type of modification/derivation, degree and type of adulteration and the like. Some fragrance will be useful in amounts from about 0.001 to about 1.0 part per 100 parts of the adhesive composition. Typically, the amount of an essential oil composition used in an adhesive in accordance with the invention will be will be from 0.05 to about 15 weight percent, more preferably between about 2 and about 12 weight percent.

The resulting adhesives of the present invention are characterized by their ability to provide a fragrance and a durable bond to a nonwoven or tissue article and otherwise meet the unique requirements of the application, such flexibility, non-staining, and machinable viscosity.

The adhesive is applied to a substrate while in its molten state and cooled to harden the adhesive layer. The adhesive product can be applied to a substrate such as a nonwoven article by a variety of methods including coating or spraying in an amount sufficient to cause the article to adhere to another substrate such as tissue, nonwoven, or an unrelated material such as a low density polyolefin or other conventionally employed substrates.

The invention is further illustrated by the following non-limiting example.

### EXAMPLE

The following example illustrates the production of hot melt adhesives comprising an essential oil. All parts are by weight and all temperatures in degree Celsius unless otherwise noted. Odor was determined using a comparison test between a control sample with no fragrance and a fragranced sample. The odor is described by it intensity and smell.

Adhesive Samples A, B and C having the compositions shown in Table 1 were prepared using techniques known in the art. An exemplary procedure involves placing approximately half of the total tackifying resin in a jacketed mixing kettle, which is equipped with rotors, and raising the temperature to a range from about 100° C. to 200° C. The precise temperature utilized depends on the softening point of the particular tackifying resin. When the resin has melted, stirring is initiated and the block polymer and stabilizer are added together with any optional additives whose presence may be desired. Mixing and heating are continued until a smooth homogeneous mass is obtained.

TABLE 1

| Adhesive | | | |
|---|---|---|---|
| Composition | Sample A | Sample B | Sample C |
| Stereon 841A | 12 | 12 | 12 |
| Kraton D1124K | 8 | 8 | 8 |
| Escorez ECR 179EX | 57.6 | 57.6 | 57.6 |
| Calsol 5550 | 18.7 | 15.7 | 11.7 |
| Lavender fragrance | 3 | 6 | 10 |
| #7-139-2 | | | |
| Naurgard HM22 | 0.2 | 0.2 | 0.2 |
| Irgafos 168 | 0.5 | 0.5 | 0.5 |

Stereon 841A (Firestone Synthetic Rubber & Latex) - SBS, 43%, melt flow index of 12, linear
Kraton D1124K (Kraton Polymers) - SIS, 30% styrene, melt flow index of 5.7, branched architecture
Escorez ECR 179EX (Exxon Chemical Co.) - tackifying resin
Calsol 5550 (Calumet Lubricants Co.) - hydrogenated aliphatic/aromatic maphtenenic process oil
Lavender fragrance #7-139-2 (Essential Ingredients) - natural botanical lavender fragrance and mineral oil having a flash point >300° F. (approx. 320° F.)
Naurgard HM22 (Uniroyal Chemical Co.) - antioxidant (hindered phenol/amine)
Irgafos 168 (Ciba Specialty Corporation) - lantioxidant (phosphite)

A panel of eight individuals were presented with a series of absorbent article samples containing adhesive Samples A. B and C. The samples are then evaluated by the panelists. All fragranced samples were perceived as having a moderate lavender fragrance (i.e. refreshing, herbaceous and sweet, imparting a sense of “clean”). After extended periods of time, the presence of the fragrance was still noticeable.

Many modifications and variations of this invention can be made without departing from its spirit and scope, as will be apparent to those skilled in the art. The specific embodiments described herein are offered by way of example only, and the invention is to be limited only by the terms of the appended claims, along with the full scope of equivalents to which such claims are entitled.

1. A hot melt adhesive comprising an effective amount of a scented material having a flashpoint of greater than 100° F.

2. The adhesive of claim 1 wherein the scented material has a flashpoint of greater than 200° F.

3. The adhesive of claim 2 wherein the scented material has a flashpoint of greater than 300° F.

4. The adhesive of claim 3 wherein the scented material comprises an essential oil.

5. The adhesive of claim 1 wherein the essential material is selected from the group consisting of achillea oil, anise oil, balm mint oil, basil oil, bee balm oil, bergamot oil, birch oil, bitter almond oil, bitter orange oil, calendula oil, California nutmeg oil, caraway oil, cardamom oil, chamomile oil, cinnamon oil, clary oil, cloveleaf oil, clove oil, coriander oil, cypress oil, eucalyptus oil, fennel oil, gardenia oil, geranium oil, ginger oil, grapefruit oil, hops oil, hyptis oil, indigo bush oil, jasmine oil, juniper oil, kiwi oil, laurel oil, lavender oil, lemongrass oil, lemon oil, linden oil, lovage oil, mandarin orange oil, matricaria oil, musk rose oil, nutmeg oil, olibanum, orange flower oil, orange oil, patchouli oil, pennyroyal oil, peppermint oil, pine oil, pine tar oil, rose hips oil, rosemary oil, rose oil, rue oil, sage oil, sambucus oil, sandalwood oil, sassafras oil, silver fir oil, spearmint oil, sweet marjoram oil, sweet violet oil, tar oil, tea tree oil, thulasia oil, thyme oil, turmeric oil, wild mint oil, yarrow oil, ylang ylang oil,

6. The adhesive of claim 5 wherein the essential oil is lavender oil.

7. The adhesive of claim 6 wherein the lavender oil comprises at least one component which has been modified.

8. The adhesive of claim 6 wherein the lavender oil comprises at least one synthetic component.

9. The adhesive of claim 6 wherein the lavender oil has a closed cup flash point greater than 300° F.

10. The adhesive of claim 1 which is a pressure sensitive adhesive.

11. The adhesive of claim 1 comprising an adhesive polymer selected from the group consisting of styrene butadiene styrene polymers, styrene isoprene stryrene polymers and mixtures thereof.

12. The adhesive of claim 11 wherein the scented material comprises an essential oil.

13. The adhesive of claim 12 wherein the scented material comprises lavender oil.

14. The adhesive of claim 13 wherein the scented material further comprises a mineral oil.

15. The adhesive of claim 14 wherein the scented material has a closed cup flash point greater than 300° F.
